(19) United States
(12) Patent Application Publication
(10) Pub. No.: US 2026/0308275 A1
(43) Pub. Date: Oct. 8, 2026

(54) HYBRID TRANSIENT TRANSFECTION USING TRANSPOSASE FOR HIGH YIELD PRODUCT PRODUCTION

(71) Applicants: LONZA BIOLOGICS PLC, Slough, Berkshire (GB); LONZA HOUSTON, INC., Pearland, TX (US)

(72) Inventors: Peter M. O’CALLAGHAN, Slough, Berkshire (GB); Bernadette SWEENEY, Slough, Berkshire (GB); James HARVEY, Slough, Berkshire (GB); Devarshi KAPADIA, Pearland, TX (US)

(21) Appl. No.: 19/482,323

(22) PCT Filed: May 9, 2024
(86) PCT No.: PCT/US2024/028583

§ 371 Date: Nov. 7, 2025

Related U.S. Application Data
(60) Provisional application No. 63/501,921, filed on May 12, 2023.

(51) Int. Cl. C07K 16/32; C12N 15/86

(52) U.S. Cl. CPC C07K 16/32; C12N 15/86; C07K 2317/24; C12N 2740/15043; C12N 2740/15051; C12N 2750/14143; C12N 2750/14151

(57) ABSTRACT

A method of producing a protein of interest, comprising: providing a host cell, a vector comprising a nucleic acid sequence encoding the protein of interest, and a transposase; transfecting the host cell using the vector and the transposase to obtain a transfected cell; culturing the transfected cell to form a culture; and harvesting the culture to obtain the protein of interest from the harvested culture, wherein cells of the culture are not passaged prior to harvesting the culture.

### FIELD OF THE INVENTION

The present disclosure provides methods for producing a protein of interest by hybrid transient transfection with transposase. By combining transposase with transient transfection, the protein of interest can be produced at a high yield within a short period of time.

### BACKGROUND OF THE INVENTION

Transient gene expression (TGE) is useful for generating small amounts of gene product(s) in a short and limited time span. In this approach, an expression vector enters a host cell line, but highly stringent selection pressure for stable integration of the vector DNA into the host cell line genome is not applied. On the other hand, stable pool gene expression is useful for generating large amount of gene product(s) over a prolonged time span, where stringent selection is applied so that only cells that have incorporated the expression vector into transcriptionally-active regions of the genome can survive. Both TGE and stable gene expression have their limitations.

### SUMMARY OF THE INVENTION

In some embodiments, provided herein is a method of producing a protein of interest, comprising: providing a host cell, a vector comprising a nucleic acid sequence encoding the protein of interest, and a transposase; transfecting the host cell using the vector and the transposase to obtain a transfected cell; culturing the transfected cell to form a culture; and harvesting the culture to obtain the protein of interest from the harvested culture. The method takes advantages of transient gene expression by expressing the protein of interest within a short period of time without the stringent selection and passaging steps, and takes advantages of stable pool expression by providing a high yield of the protein of interest.

### BRIEF DESCRIPTION OF THE DRAWINGS

FIG. 1 shows a glutamine synthetase PIGGYBAC® transient expression platform according to some embodiments of the disclosure, which achieves extended production of a protein of interest and high titers of the protein of interest.

FIG. 2 shows a polyethylenimine (PEI) transfection procedure according to some embodiments of the present disclosure.

FIG. 3A shows product titers from three different product expression platforms according to some embodiments of the present disclosure.

FIG. 3B shows bYlok bsAb titers using standard transient expression and glutamine synthetase PIGGYBAC® transient expression according to some embodiments of the present disclosure.

FIG. 4A shows isoforms of the cB72.3 product using glutamine synthetase PIGGYBAC® stable pool expression and glutamine synthetase PIGGYBAC® transient expression according to some embodiments of the present disclosure.

FIG. 4B shows aggregation of the cB72.3 product using glutamine synthetase PIGGYBAC® stable pool expression and glutamine synthetase PIGGYBAC® transient expression according to some embodiments of the present disclosure.

FIG. 4C shows N-glycans of the cB72.3 product using glutamine synthetase PIGGYBAC® stable pool expression and glutamine synthetase PIGGYBAC® transient expression according to some embodiments of the present disclosure.

FIG. 5A shows expression of Trastuzumab using standard transient expression and glutamine synthetase PIGGYBAC® transient expression according to some embodiments of the present disclosure.

FIG. 5B shows expression of AMS002 using standard transient expression and glutamine synthetase PIGGYBAC® transient expression according to some embodiments of the present disclosure.

FIG. 5C shows expression of bsAb using standard transient expression and glutamine synthetase PIGGYBAC® transient expression according to some embodiments of the present disclosure.

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value. Typically, the term is meant to encompass approximately or less than 1%, 2%, 3%, 4%, 5%, 6%, 7%, 8%, 9%, 10%, 11%, 12%, 13%, 14%, 15%, 16%, 117% 18%, 19% or 20% variability depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps.

As used herein, “nucleic acid,” “nucleic acid molecule,” or “oligonucleotide” means a polymeric compound comprising covalently linked nucleotides. The term “nucleic acid” includes polyribonucleic acid (RNA) and polydeoxyribonucleic acid (DNA), both of which may be single- or double-stranded. DNA includes, but is not limited to, complimentary DNA (cDNA), genomic DNA, plasmid or vector DNA, and synthetic DNA. RNA includes, but is not limited to, mRNA, tRNA, rRNA, snRNA, microRNA, miRNA, or MIRNA.

A “gene” as used herein refers to an assembly of nucleotides that encode a polypeptide, and includes cDNA and genomic DNA nucleic acid molecules. “Gene” also refers to a nucleic acid fragment that can act as a regulatory sequence preceding (5′ non-coding sequences) and following (3′ non-coding sequences) the coding sequence. In some embodiments, genes are integrated with multiple copies. In some embodiments, genes are integrated at predefined copy numbers.

“Transfection” as used herein means the introduction of an exogenous nucleic acid molecule, including a vector, into a cell. A “transfected” cell comprises an exogenous nucleic acid molecule inside the cell and a “transformed” cell is one in which the exogenous nucleic acid molecule within the cell induces a phenotypic change in the cell.

The term “transfection” covers a range of techniques that are used to introduce the gene(s) of interest into a host cell line. These techniques include, for example, liposome-based transfection (where a transfection reagent is mixed with the DNA to form a “liposome”), electroporation, and the use of cationic polymers. In some embodiments, the cationic polymer used for transfection is the cationic polymer polyethylenimine (PEI). PEI polyplexes combine with DNA to form positively charged particles that bind to the negatively-charged cell surface before being endocytosed. Upon release into the cell cytoplasm, the DNA can migrate to the nucleus where gene expression can be initiated. PEI exists in both branched and linear forms of different molecular masses, with transfection efficiency varying greatly between the different forms. In some embodiments, the PEI reagent used in the present disclosure has a linear format of 40,000 MW. In some embodiments, the transfection procedure can be further optimized to increase efficiency and reduce toxicity.

In certain aspects, the present disclosure provides a method of producing a protein of interest or another types of gene product. In some embodiments, the method does not require stringent selection and complicated passaging, and can produce the protein of interest at high yield within a short period of time.

In some embodiments, the method comprising: providing a host cell, a vector comprising a nucleic acid sequence encoding the protein of interest, and a transposase; transfecting the host cell using the vector and the transposase to obtain a transfected cell; culturing the transfected cell to form a culture; and harvesting the culture to obtain the protein of interest from the harvested culture, wherein cells of the culture are not passaged prior to harvesting the culture.

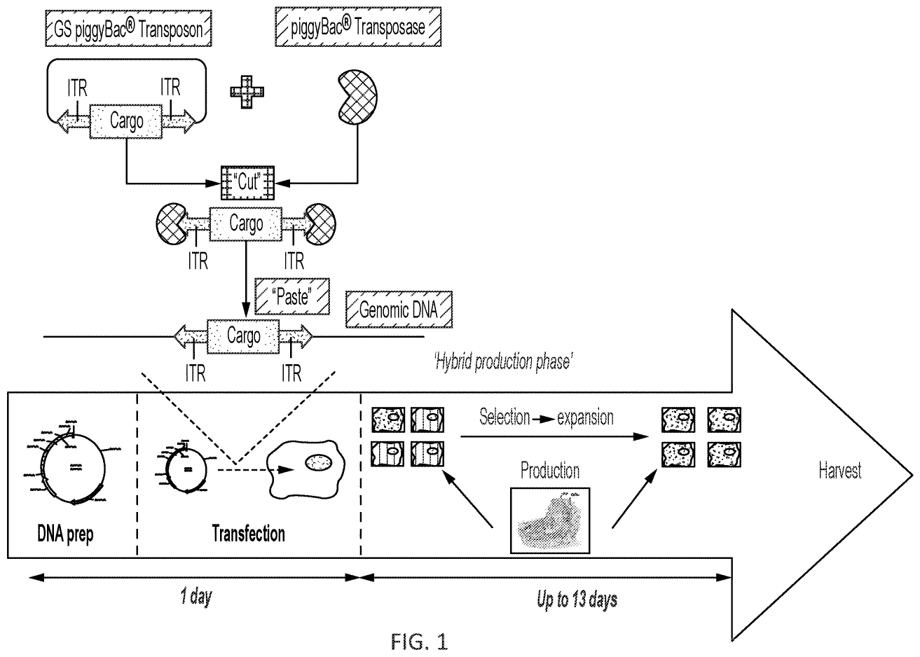

FIG. 1 shows a method of producing a protein of interest according to some embodiments of the disclosure. As shown in FIG. 1, a vector (DNA prep) containing the gene of interest and a transposase are used to co-transfect host cells. After the transfection, the transfected cells are cultured, and a period for culturing the transfected cells and producing the protein of interest is named a hybrid production phase. During this period, the transfected cells are selectively expanded, and production of the protein of interest from the gene of interest occurs. The selection may be performed under a condition that is less stringent than that is required for stable pool selection. In this hybrid production phase, there is no interruption of the process post-transfection by passaging, and there is no need to remove cells from the culture. This contrasts with a stable pool construction process where transposases are normally used, whereby a distinct culture selection phase occurs to expand transfected cells, prior to at least one subsequent sub-culturing step, before inoculation of a production vessel for dedicated synthesis of the recombinant protein. Instead, cell harvesting and protein collection can begin immediately (i.e., with minutes to hours to less than 1 day) following the end of the transfection procedure. There is no need, requirement, or desire for passaging of the cells. This means that any protein product produced immediately following transfection may end up being harvested at the end of the hybrid production phase. This contrasts with a stable pool process, whereby material generated and secreted into the growth media during the post-transfection selection phase may be lost as a result of the subsequent sub-culturing step(s). In this hybrid process no protein product generated after transfection is lost from the end-point harvest material as a result of an intermediate sub-culture step. As used herein “passaging” refers to the procedure of harvesting cells from a culture, transferring the cells to one or more culture vessels with fresh growth medium, and using those cells to start new cultures, and is also known as subculturing. As is known in the art, passaging of cells allows a subset of cells to then continue to propagate and grow. In such instances, these passaged cells and their progeny are thus a different cell lineage from the original transfected cell line.

As shown in FIG. 1, in embodiments the vector can be a glutamine synthetase PIGGYBAC® Transposon, and the transposase can be a PIGGYBAC® transposase (including a hyperactive super PIGGYBAC®). The vector PIGGYBAC® Transposon carries a cargo (the gene of interest encoding the protein of interest), and inverted terminal repeat (ITR) sequence flanking the cargo. The ITR sequences are recognizable by the hyperactive PIGGYBAC® transposase. By using the hyperactive glutamine synthetase PIGGYBAC® transposase, the disclosure can integrate the stable pool post transfection “selection-expansion-production” phase into a “hybrid” production phase to support high titers across a range of protein formats. The transfected vector containing the product suitably have the PIGGYBAC® ITRs. The transposase can be delivered into the cells as mRNA, as a separate piece of non-GS vector DNA, or can even be contained with the GS transposon along with the product genes.

In the embodiments, glutamine (e.g., about 3-10 mM, suitably 6 mM) is added in the medium before transfection, while the medium after transfection does not include the glutamine, so as to have a selection function. In some embodiments, the selection is less stringent, and about 0.5 mM to about 5 mM glutamine can be added to the medium after transfection. In some embodiments, the added glutamine is at a concentration of about 1 mM to 4 mM. In some embodiments, the added glutamine is at a concentration of about 3 mM. The reduced or eliminated glutamine media can promote the growth of productive cells containing the expression vector, which may allow culture durations to be extended to achieve higher product titers.

In some embodiments, methionine sulfoxamine (MSX) is not added in the medium after transfection in this glutamine synthetase-based transient expression system. In contrast, MSX is added in the medium after transfection in the glutamine synthetase-based stable pool expression system. As a further comparison, the requirement for glutamine-containing media, such as a media having 6 mM glutamine, is normally retained after transient transfection, as highly efficient integration of the glutamine synthetase vector into the host cell genome may not have occurred.

Host cells or organisms that express exogenous nucleic acid molecules or fragments are referred to as “recombinant,” “transformed,” or “transgenic” organisms. In some embodiments, the host cell is a cell suitable for expressing exogenous nucleic acid molecules or fragments. Suitably, the host cells that can be utilized in the various methods described herein are mammalian cells and cell lines or cultures. As used herein, the term “mammalian cell” includes cells from any member of the order Mammalia, such as, for example, human cells, mouse cells, rat cells, monkey cells, hamster cells, and the like. In some embodiments, the cell is a mouse cell, a human cell, a Chinese hamster ovary (CHO) cell, a CHOK1 cell, a CHO-DXB11 cell, a CHO-DG44 cell, a CHOKlSV cell including all variants (e.g., POTELLIGENT®, Lonza, Slough, UK), a CHOKISV GS-KO (glutamine synthetase knockout) cell including all variants (e.g., Xceed® Lonza, Slough, UK), a baby hamster kidney (BHK) cell. Exemplary human cells include human embryonic kidney (HEK) cells, such as HEK-293, a HeLa cell, or a HT1080 cell. In some embodiments, the protein of interest of the present disclosure is produced from HEK-293 cells, Human Caucasian colon adenocarcinoma HT-29 cells, or mesenchymal stem cells (MSCs). In some embodiments, the protein of interest of the present disclosure is produced by the CHOK1SV GS-KO® host cell. In some embodiments, the host cell is a glutamine synthetase Xceed® cell.

Mammalian cells include mammalian cell cultures which can be either adherent cultures or suspension cultures. Adherent cultures refer to cells that are grown on a substrate surface, for example a plastic plate, dish or other suitable cell culture growth platform, and may be anchorage dependent. Suspension cultures refer to cells that can be maintained in, for example, culture flasks or large suspension vats, which allows for a large surface area for gas and nutrient exchange. Suspension cell cultures often utilize a stirring or agitation mechanism to provide appropriate mixing. Media and conditions for maintaining cells in suspension are generally known in the art. An exemplary suspension cell culture includes human embryonic kidney (HEK293) clonal cells.

In some embodiments, the host cell, such as the CHOKISV GS-KO® host cell, is pre-cultured for about 4 days to about 28 days to form a pre-culture before the transfecting step. In some embodiments, the host cell is pre-cultured for about 6 days to about 14 days. In some embodiments, the host cell is pre-cultured for about 8 days to about 10 days. In some embodiments, the host cell, such as the CHOK1SV GS-KO® host cell, is cultured in a chemically defined Chinese hamster ovary (CD CHO) medium. In some embodiments, the CD CHO medium is supplemented with glutamine before the transfecting step. In some embodiments, the CD CHO medium is substantially devoid of methionine sulfoxamine.

In some embodiments, the host cells can be cultured in other media, such as Dulbecco's Modified Eagle Medium (DMEM), Roswell Park Memorial Institute (RPMI) 1640, or minimum Essential Medium (MEM), a CD CHO medium without supplementing glutamine, or a combination thereof.

In some embodiments, the pre-cultured host cells before transfection have at least 70% cell viability. In some embodiments, the pre-cultured host cells before transfection have at least 75%, 80%, 85%, 90%, or at least 95% cell viability. In some embodiments, the pre-cultured host cells before transfection have at least 90% cell viability. In some embodiments, the cell viability is determined by trypan blue dye exclusion. In some embodiments, the trypan blue dye is a 0.4%, sterile-filtered solution from SIGMA.

In some embodiments, the method provides a vector comprising a nucleic acid sequence encoding the protein of interest. The nucleic acid sequence is also referred to as the gene of interest herein. In some embodiments, the vectors comprise an adenoviral vector, a pSV vector, a pCMV vector, a vaccinia vector, a retroviral vector, or a baculovirus vector. In some embodiments, the vector has a glutamine synthetase gene, while the host cell is a knockout of the glutamine synthetase gene. That is the host cell does not express endogenous glutamine synthetase, and the transfection of the vector into the cell provides exogenous expression of the glutamine synthetase, such that insertion of the vector to the host cell can be selected using a medium without glutamine. In some embodiments, the vector comprises hyperactive super PIGGYBAC® inverted terminal repeat (ITR) sequences. In some embodiments, the vector comprises two ITR sequences, and each side of the gene of interest is flanked by one of the two ITR sequences. The ITR sequences enable the vector compatible with PIGGYBAC® transposase. In some embodiments, the vector is a glutamine synthetase PIGGYBAC® Transposon. In some embodiments, the vector is a GSquad™ expression vector.

In some embodiments, one vector is provided, which comprises the intact gene of interest for expressing the protein of interest. In some embodiments, one vector is provided, which includes several gene fragments of the gene of interest. Each fragment of the gene of interest is used to express a part of the protein, and the expressed parts of the protein can be assembled to form the protein of interest. The assembly of the protein can be a self-assembly process. In some embodiments, one vector is provided, which includes two or more genes of interest. The vector is used to express two or more protein products simultaneously. In some embodiments, the two or more protein products are functionally related. In some embodiments, the two or more protein products can self-assemble to form a polymeric protein or a macromolecule assembly.

In some embodiments, two or more vectors are provided, each of the vectors includes a fragment of the gene of interest, and expresses a part of the protein of interest. The expressed parts of the protein of interest can be assembled or self-assemble to form the protein of interest. In some embodiments, two or more vectors are provided, each vector includes a protein of interest. The multiple vectors can be used to transfect the host cell simultaneously together with the transposase, and the transfected cell can be used to express multiple proteins of interest simultaneously. The proteins of interest can be functionally related. In some embodiments, the expressed proteins of interest are assembled or self-assembled to form a polymeric protein or a macromolecule assembly.

Exemplary proteins of interest that can be produced using the methods described herein include, for example, various antibodies, including antibody fragments and single chain antibodies, as well as other therapeutic proteins or diagnostic proteins.

In exemplary embodiments the gene of interest can encode components required for assembling virus particles (i.e., a viral gene of interest), including lentivirus and adeno-associated virus particles. Suitably, the methods described herein can provide for the production of three AAV constructs: pHelper containing E2A, E4, and VA; pRepCap containing Rep and Cap; and pAAV containing a gene of interest (GOI). In other embodiments, the methods described herein can be used to produce lentivirus constructs including Lentiviral group specific antigen (GAG) gene and a lentiviral polymerase (POL) protein; Envelope protein (usually Vesicular Stomatitis Virus Glycoprotein (VSV-G)); HIV regulator of expression of virion proteins (Rev) protein; and a gene of interest (GOI).

Suitably, the amount of the protein of interest or the gene of interest that is produced using the methods described herein is at least 20% more than a typical transient transfection system. More suitably, the amount of the protein of interest or the gene of interest that is produced using the methods described herein is at least 30% more, at least 40% more, at least 50% more, at least 60% more, at least 70% more, at least 80% more, at least 90% more, at least 100% more, at least 150% more, at least 200% more, or about 50% to about 200% more.

Various transposases can be utilized in the methods described herein. In some embodiments, the transposase comprises an RNase H-like transposase, a HUH single-stranded DNA transposase, a serine transposase, a tyrosine transposase, a glutamine synthetase PIGGYBAC® transposase, or a combination thereof. In some embodiments, the transpose comprises a hyperactive glutamine synthetase PIGGYBAC® transposase. In some embodiments, the transposase is contained in a transposase vector or plasmid, such that the transposase is encoded by the cell, rather than added as a functional enzyme directly to the cell. In other embodiments, the transposase can be delivered as mRNA.

“Transfection” as used herein means the introduction of an exogenous nucleic acid molecule, including a plasmid and/or vector, into a cell. A “transfected” cell comprises an exogenous nucleic acid molecule inside the cell and a “transformed” cell is one in which the exogenous nucleic acid molecule within the cell induces a phenotypic change in the cell. The transfected nucleic acid molecule can be integrated into the host cell's genomic DNA and/or can be maintained by the cell, temporarily or for a prolonged period of time, extra-chromosomally. In some embodiments, “transduction” means infection of mammalian cells with a viral vector, and is used interchangeably with “transfection” in the disclosure.

In some embodiments, the host cells are determined to have a high viability before the transfection. A variety of transfection techniques can be applied. In some embodiments, the transfection of the host cell is performed using polyethylenimine (PEI), lipofection, electroporation, magnetofection, microinjection, gene gun insertion, impalefection, hydrostatic pressure, or sonication. In some embodiments, the transfection is performed using PEI.

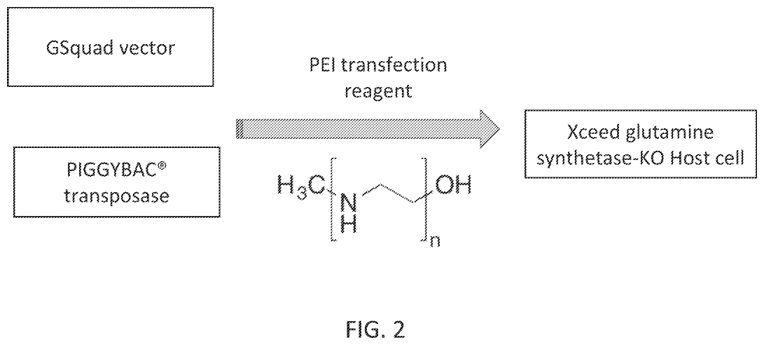

FIG. 2 shows one example of transfection, where a GSquad™ vector containing a gene of interest and a hyperactive PIGGYBAC® transposase are used together to transfect a Xceed glutamine synthetase-KO® host cell by PEI transfection reagent.

After transfection, the transfected cells are cultured in a medium, but no cell passaging takes place (i.e., no cells are removed for preparation of a further culture for production of the protein of interest). A variety of media can be used for the culturing of the transfected cells, for example, Dulbecco's Modified Eagle Medium (DMEM), Roswell Park Memorial Institute (RPMI) 1640, Minimum Essential Medium (MEM), a chemically defined Chinese hamster Ovary (CD CHO) medium. In some embodiments, the medium is CD CHO medium. In some embodiments, the vector comprises the glutamine synthetase gene, and the medium is not supplemented with glutamine. In some embodiments, the medium does not include methionine sulfoxamine (MSX). This is different from stable pool expression, where MSX is used for stringent selection of transfected cells that have the vectors stably integrated in the genome of the cells. In some embodiments, bolus fed is performed to extend production. In some embodiments, the cell culture is bolus fed at about day 3 after transfection of the host cells. In some embodiments, the cell culture is bolus fed at day 2 and day 5 after transfection of the host cells.

In some embodiments, culturing of the transfected cells acts as both selection/expansion and production. The lack of glutamine or low concentration of glutamine in the medium enables almost immediate selection of transfected cells containing the vector, where the vector may or may not be integrated in the cell genome. The transfected cells containing the vector are multiplied much faster than the cells without the vector, such that the transfected cells are expanded. Since glutamine and MSX are not added in the medium, the selection is not as stringent as selecting for a stable pool of cells. As described herein, all of the original cells present in the culture at the point of transfection are kept in the culture, and the cells are used for production of the protein of interest in an early culturing stage, rather than passaging the cells for later protein harvesting. In some embodiments, based on the host cell and the vector, other non-stringent selection criteria other than the glutamine-/MSX-medium can be used. As discussed above, the culturing includes both selection/expansion and production, and thus is also named a hybrid production phase.

In some embodiments, the transfected cells are cultured in a humid CO₂ orbital shaker incubator, using Erlenmeyer shake-flasks with vented caps. In some embodiments, the transfected cells are cultured in shake-flasks with sealed caps, provided a sterile, premixed supply of 5% CO₂ in air is available to gas the headspace of flasks or bottles during set-up of the culture and after opening.

In some embodiments, the cells and the protein of interest are produced in a bioreactor. The cells can be prepared in any suitable bioreactor (also called reactor herein) including but not limited to stirred tank, airlift, fiber, microfiber, hollow fiber, ceramic matrix, fluidized bed, fixed bed, and/or spouted bed bioreactors. As used herein, “bioreactor” can include a fermenter or fermentation unit, or any other reaction vessel and the terms “bioreactor” and “reactor” are used interchangeably with “fermenter.” The term fermenter or fermentation refers to both microbial and mammalian cultures. For example, in some aspects, an example bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO₂ levels, maintenance of pH level, agitation (e.g., stirring), and/or cleaning/sterilizing. Example reactor units, such as a fermentation unit, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. In various embodiments, the bioreactor can be suitable for batch, semi fed-batch, fed-batch, perfusion, and/or a continuous fermentation process. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass.

As discussed above, the protein production starts in the early stage of the cell culturing. In some embodiments, the transfection occurs on day 1, and the harvesting of the cells can begin on day 2. There is no passage of the cells after transfection, and there is no need to remove cells from the culture other than harvesting. In some embodiments, the harvesting continues from day 2 to at least day 14. In some embodiments, the harvesting continues from day 2 to about 8 to 12 days.

In some embodiments, the harvesting is performed once at the end of the culturing process, and the harvesting is performed at day, 1, day 2, day 3, day 4, day 5, day 6, day 7, day 8, day 9, day 10, day 11, day 12, day 13 or day 14 and can continue for additional days up to additional weeks.

The hybrid production phase takes advantage of transient expression and stable pool expression. Compared to transient expression, the production can be extended to about 13 days after transfection, so as to obtain high titers. Compared to stable pool expression, the high stringent selection and passage process is avoided, no cells (i.e., less than 10%, less than 5%, or less than 1% of the original cell culture) are removed from the cell culture, and the production period is much shorter.

In additional aspects, the present disclosure provides a method of producing a protein of interest. In some embodiments, the method comprises: providing a host cell, a vector comprising a nucleic acid sequence encoding the protein of interest, and a transposase; transfecting the host cell using the vector and the transposase to obtain a transfected cell; culturing the transfected cell to form a culture; and harvesting the culture to obtain the protein of interest from the harvested culture, wherein cells of the culture are of the same cell lineage as the transfected cell. In embodiments, the cells in the culture are of the same cell lineage as the transected cells because no stringent selection and no cell passage are required. Thus, cells “of the same cell lineage” refer to the original transfected cells not being removed for separate cell culture and propagation prior to protein harvesting.

### Embodiments

In a first embodiment, provided herein is a method of producing a protein of interest, comprising: providing a host cell, a vector comprising a nucleic acid sequence encoding the protein of interest, and a transposase; transfecting the host cell using the vector and the transposase to obtain a transfected cell; culturing the transfected cell to form a culture; and harvesting the culture to obtain the protein of interest from the harvested culture, wherein cells of the culture are not passaged prior to harvesting the culture.

Embodiment 2 includes the method of embodiment 1, wherein the host cell comprises a mammalian cell line.

Embodiment 3 includes the method of embodiment 2, wherein the mammalian cell line comprises a Chinese hamster ovary (CHO) cell line, a baby hamster kidney (BHK) cell line, a murine myeloma cell line, a human embryonic kidney cell line (HEK) cell line, or a HeLa cell line.

Embodiment 4 includes the method of embodiment 1, wherein the host cell comprises a CHOKISV glutamine synthetase-knock out cell line.

Embodiment 5 includes the method of any of embodiments 1 to 4, wherein the host cell is pre-cultured for about 4 days to about 28 days to form a pre-culture before the transfecting.

Embodiment 6 includes the method of embodiment 5, wherein the host cell is pre-cultured in a chemically defined Chinese hamster Ovary (CD CHO) medium supplemented with 6 mM glutamine before transfection.

Embodiment 7 includes the method of embodiments 5 or 6, wherein the pre-culture has at least 90% cell viability.

Embodiment 8 includes the method of any of embodiments 1 to 7, wherein the vector comprises an adenoviral vector, a pSV vector, a pCMV vector, a vaccinia vector, a retroviral vector, or a baculovirus vector.

Embodiment 9 includes the method of any of embodiments 1 to 8, wherein the vector comprises a glutamine synthetase gene.

Embodiment 10 includes the method of embodiment 9, wherein the vector comprises a hyperactive super PIGGYBAC® (glutamine synthetase PIGGYBAC®) inverted terminal repeat (ITR) sequence.

Embodiment 11 includes the method of any of embodiments 1 to 10, wherein the vector comprises one to about 5 nucleic acid sequences encoding the protein of interest, or the vector comprises one to about 5 different vectors comprising the nucleic acid sequence.

Embodiment 12 includes the method of any one of embodiments 1 to 11, wherein the transposase comprises an RNase H-like transposase, a HUH single-stranded DNA transposase, a serine transposase, a tyrosine transposase, a glutamine synthetase PIGGYBAC® transposase, or a combination thereof.

Embodiment 13 includes the method of embodiment 12, wherein the transposase comprises a glutamine synthetase PIGGYBAC® transposase.

Embodiment 14 includes the method of any of embodiments 1 to 13, wherein the transposase is contained in a transposase vector.

Embodiment 15 includes the method of any of embodiments 1 to 14, wherein transfecting the host cell is performed using polyethylenimine (PEI), lipofection, electroporation, magnetofection, microinjection, gene gun insertion, impalefection, hydrostatic pressure, or sonication.

Embodiment 16 includes the method of embodiment 15, wherein transfecting the host cell is performed using PEI.

Embodiment 17 includes the method of any of embodiments 1 to 16, wherein the transfected cells are cultured in a medium, and the medium comprises Dulbecco's Modified Eagle Medium (DMEM), Roswell Park Memorial Institute (RPMI) 1640, Minimum essential medium (MEM), a chemically defined Chinese hamster Ovary (CD CHO) medium without supplementing glutamine, or a combination thereof.

Embodiment 18 includes the method of embodiment 17, wherein the medium comprises CD CHO medium without supplementing glutamine.

Embodiment 19 includes the method of embodiment 18, wherein the culture is devoid of methionine sulfoxamine (MSX).

Embodiment 20 includes the method of any of embodiments 1 to 19, wherein the culture is bolus fed at day 2 and day 5 after transfecting the host cell.

Embodiment 21 includes the method of any of embodiments 1 to 20, wherein transfecting occurs on day 1 and harvesting begins on day 2 of the method.

Embodiment 22 includes the method of embodiment 21, wherein harvesting continues from day 2 to at least day 14 of the method.

Embodiment 23 provides a method of producing a protein of interest, comprising: providing a host cell, a vector comprising a nucleic acid sequence encoding the protein of interest, and a transposase; transfecting the host cell using the vector and the transposase to obtain a transfected cell; culturing the transfected cell to form a culture; and harvesting the culture to obtain the protein of interest from the harvested culture, wherein cells of the culture are of the same cell lineage as the transfected cell.

Embodiment 24 includes the method of embodiment 23, wherein the host cell comprises a mammalian cell line.

Embodiment 25 includes the method of embodiment 24, wherein the mammalian cell line comprises a Chinese hamster ovary (CHO) cell line, a baby hamster kidney (BHK) cell line, a murine myeloma cell line, a human embryonic kidney cell line (HEK) cell line, or a HeLa cell line.

Embodiment 26 includes the method of embodiment 23, wherein the host cell comprises a CHOKlSV glutamine synthetase-knock out cell line.

Embodiment 27 includes the method of any of embodiments 23 to 26, wherein the host cell is pre-cultured for about 4 days to about 28 days to form a pre-culture before the transfecting.

Embodiment 28 includes the method of embodiment 27, wherein the host cell is pre-cultured in a chemically defined Chinese hamster Ovary (CD CHO) medium supplemented with 6 mM glutamine before transfection.

Embodiment 29 includes the method of embodiments 27 or 28, wherein the pre-culture has at least 90% cell viability.

Embodiment 30 includes the method of any of embodiments 23 to 29, wherein the vector comprises an adenoviral vector, a pSV vector, a pCMV vector, a vaccinia vector, a retroviral vector, or a baculovirus vector.

Embodiment 31 includes the method of any of embodiments 23 to 30, wherein the vector comprises a glutamine synthetase gene.

Embodiment 32 includes the method of embodiment 31, wherein the vector comprises a hyperactive super PIGGYBAC® (glutamine synthetase PIGGYBAC®) inverted terminal repeat (ITR) sequence.

Embodiment 33 includes the method of any of embodiments 23 to 32, wherein the vector comprises one to about 5 nucleic acid sequences encoding the protein of interest, or the vector comprises one to about 5 different vectors comprising the nucleic acid sequence.

Embodiment 34 includes the method of any one of embodiments 23 to 33, wherein the transposase comprises an RNase H-like transposase, a HUH single-stranded DNA transposase, a serine transposase, a tyrosine transposase, a glutamine synthetase PIGGYBAC® transposase, or a combination thereof.

Embodiment 35 includes the method of embodiment 34, wherein the transposase comprises a glutamine synthetase PIGGYBAC® transposase.

Embodiment 36 includes the method of any of embodiments 23 to 35, wherein the transposase is contained in a transposase vector.

Embodiment 37 includes the method of any of embodiments 23 to 36, wherein transfecting the host cell is performed using polyethylenimine (PEI), lipofection, electroporation, magnetofection, microinjection, gene gun insertion, impalefection, hydrostatic pressure, or sonication.

Embodiment 38 includes the method of embodiment 37, wherein transfecting the host cell is performed using PEI.

Embodiment 39 includes the method of any of embodiments 23 to 38, wherein the transfected cells are cultured in a medium, and the medium comprises Dulbecco's Modified Eagle Medium (DMEM), Roswell Park Memorial Institute (RPMI) 1640, Minimum essential medium (MEM), a chemically defined Chinese hamster Ovary (CD CHO) medium without supplementing glutamine, or a combination thereof.

Embodiment 40 includes the method of embodiment 39, wherein the medium comprises CD CHO medium without supplementing glutamine.

Embodiment 41 includes the method of embodiment 40, wherein the culture is devoid of methionine sulfoxamine (MSX).

Embodiment 42 includes the method of any of embodiments 23 to 41, wherein the culture is bolus fed at day 2 and day 5 after transfecting the host cell.

Embodiment 43 includes the method of any of embodiments 23 to 42, wherein transfecting occurs on day 1 and harvesting begins on day 2 of the method.

Embodiment 44 includes the method of embodiment 43, wherein harvesting continues from day 2 to at least day 14 of the method.

Embodiment 45 is a method of producing a viral gene of interest, comprising: providing a host cell, a vector comprising a nucleic acid sequence encoding the viral gene of interest, and a transposase; transfecting the host cell using the vector and the transposase to obtain a transfected cell; culturing the transfected cell to form a culture; and harvesting the culture to obtain the viral gene of interest from the harvested culture, wherein cells of the culture are not passaged prior to harvesting the culture.

Embodiment 46 includes the method of claim 45, wherein the viral gene of interest produces an AAV virus particle or a lentivirus particle.

### EXAMPLES

### Example 1: PEI Transient Expression

In this example, a PEI baseline transient expression is conducted according to certain embodiments of the present disclosure. The time of the procedure is show in the following Table 1:

| | | | | | | | |
|---|---|---|---|---|---|---|---|
| Day −1 | Day 0 | Day 1 | Day 2 | . . . | Day 8-10 | . . . | Day 14 |

| | | | |
|---|---|---|---|
| PEI | Seed | Transfection | Harvest |
| Baseline | cells | | |

### I. Preparation Before the Day of Transfection (day −1)

1. Revive the CHOKISV GS-KO® Host Cell Line from Cryopreservation and Perform routine culture until the day before transfection. CHOKISV GS-KO® host cells are suitably in culture for no less than 4 days but no longer than 28 days before transfection. The culture can have a viability of greater than 90% before transfection (as measured by trypan blue dye exclusion). Cultures are grown leading up to transfection using the medium CD CHO/6 mM L-glutamine. The CD CHO can be, for example Thermo Fisher Scientific, catalogue number 10743-029.
2. Prepare the vector constructs for transfection. The uncut vector DNA is suspended at 1000 μg/mL in sterile Tris-EDTA (TE) buffer. Further, for products with two, three or four genes, a single double gene vector (DGV), triple gene vector (TGV) or quadruple gene vector (QGV) can be used, or multiple Part Vectors can be co-transfected for transient expression.
3. On the day before transfection, perform subculture of the host cell line:
3.1 Pre-warm the appropriate growth medium (CD CHO+6 mM glutamine) to 35.5-37.0° C.
3.2 Aseptically remove a sample of cell suspension from the CHOK1SV GS-KO® cells to be counted using a sterile serological pipette.
3.3 Use a haemocytometer or automated cell counting device to count the cells, using trypan blue dye or other method to assess cell viability. Use the cell count and viability data to determine the cell concentration and culture viability.
3.4 For each 20 mL of transiently transfected culture required, inoculate 30 mL of new culture at a target concentration of 1.0×10⁶ viable cells/mL. A range of recommended culture volumes is given in Table 2.
3.5 If using vented-caps, re-fit the cap and place the new culture into a humid CO₂ orbital shaker incubator set at 35.5-37.0° C., 140±5 rpm, >85% relative humidity and 5% CO₂ in air.
3.6 If using sealed-caps, use a sterile, premixed supply of 5% CO₂ in air to gas out the headspace before re-fitting the cap and placing the new culture into an orbital shaker incubator set at 35.5-37.0° C. and 140±5 rpm.

TABLE 2
shows the culture volumes specific to
transient transfection of Example 1.

| | Culture Volume to Inoculate |
|---|---|
| Final Culture Volume | the Day Before Transfection (Day −1) |
| Required (mL) | at 1.0 × 10⁶ viable cells/mL [a] (mL) |
| 20 | 30 |
| 40 | 60 |
| 100 | 150 |
| 120 | 300 |

### II. Transfection (Day 0)

On the day of transfection (one day after subculture, considered as Day 0 in this procedure):
1. Calculate the total amounts of medium, PEI, sodium acetate, cells and glutamine synthetase vector DNA needed for transfection.
1.1 For a single standard transfection at a 20 mL scale, a total of 20×10⁶ viable cells, 40 μg GS vector DNA encoding the product genes, 100 μL PEI (Transporter 5, Polysciences catalogue number 26008-5), and 67 μL of sodium acetate (3M, pH 5.2, Lonza Bioscience catalogue number 51203) are needed.
1.2 Transfections may be scaled up or down, but the relative ratios of reagents per mL of culture are suitably maintained as shown in Table 3.

TABLE 3
Ratios of reagents at transfection

| | Amount per mL of |
|---|---|
| Component | transfected culture |
| CHOK1SV GS-KO ® host cells | 1 × 10⁶ viable cells |
| Transporter 5 ® Transfection Reagent | 5 μL |
| Circular plasmid DNA (stock 1 mg/mL) | 2 μg |
| Sodium Acetate (3M) | 3.4 μL (10 mM final conc.) |

Please note that the process described above is for transfection of a single glutamine synthetase vector carrying up to four product genes. However, if two vectors (e.g., for co-expression of multiple Part Vectors) are co-transfected, the plasmid DNA quantity is shared between the two vectors. For example, for a 20 mL transfection, 20 μg of each vector is transfected. Too little DNA means that the transfection is inefficient, while too much DNA can become toxic to the cells.
2. Aseptically prepare the appropriate volume of CD CHO with 6 mM L-glutamine and pre-warm to 35.5-37.0° C.
3. Using a sterile serological pipette, aseptically remove a sample of cell suspension from the CHOK1SV GS-KO® culture that had been inoculated at 1.0×10⁶ viable cells/mL the day before.
4. Use a haemocytometer or automated cell counting device to count the cells, using trypan blue dye or other methods to assess cell viability. Use the cell count and viability data to determine the cell concentration, culture viability and total number of viable cells. Please note that the culture suitably should be at least 90% viable before transfection. Poor viability reduces the transfection efficiency.
5. Calculate the volume of culture needed to give the required number of cells for transfection (20×10⁶ viable cells per 20 mL transfection). Please note that cells can be lost during the centrifuging and washing steps. It is therefore advisable in the next step to centrifuge up to 2-fold more cells than are required and to centrifuge cells at 200×g.
5.1 Aseptically remove the calculated volume of culture (from the previous step) and centrifuge at 200×g for 5 minutes.
5.2 Remove the supernatant, dislodge the pellet by gently tapping the base of the tube, and disperse the cell pellet in a sufficient volume of pre-warmed CD CHO+6 mM L-glutamine to give a concentration 1.0×10⁶ viable cells/mL.
5.3 Determine the viable cell concentration and adjust to a final concentration of 1.0×10⁶ viable cells/mL using CD CHO+6 mM L-glutamine

The cell suspension is incubated at ambient temperature whilst the transfections are performed for a maximum of 30 minutes. Further, it is important that the cell suspension prepared is maintained at ambient temperature for a maximum of 30 minutes and not at an elevated temperature. At high cell concentrations, cells maintained at 37° C. will quickly become oxygen depleted, resulting in rapid cell death.
6. For a single standard transfection, transfer 20 mL of the cell suspension from the previous step into a 125 mL Erlenmeyer flask.
7. To the flask add the following in the stated order, shaking vigorously after each addition:
7.1 40 μL (=40 μg) of sterile circular plasmid DNA at a concentration of 1 mg/mL;
7.2 100 μL of PEI, and
7.3 67 μL of sodium acetate.
8. Place the cells back into the humid CO₂ orbital shaker incubator set at 35.5-37.0° C., 140±5 rpm, >85% relative humidity and 5% CO₂ in air for 4 hours.
9. After 4 hours, move cells into a shaking incubator at 32° C., 5% CO₂, 85% humidity and shaking at 140 rpm, or lower the temperature of the existing incubator to 32° C. If using sealed-caps, use a sterile, premixed supply of 5% CO₂ in air to gas out the headspace before re-fitting the cap and placing the new culture into an orbital shaker incubator set at 32° C. and 140±5 rpm.
10. Incubate the transfected cells in the shaking incubator for between 8-10 days. The cultures may be periodically monitored for growth and viability with the goal of harvesting before the average viability drops below 90%.
11. Harvest the cultures using a suitable method, remove samples from the culture vessels, clarify and store the culture supernatant at an appropriate temperature for further assays. Please note that the shift to 32° C. can be made at any point between 4 and 24 hours post transfection to enable a better fit with laboratory schedules. However, the temperature shift must not be made before at least 4 hours.

### Example 2: GS PIGGYBAC® PEI Transient/Transposase Transience Expression

In this example, hyperactive GS PIGGYBAC® transposase enzyme is used in transient gene expression to maximize final product yield. Example 2 has similarities to stable pool construction process, but also has key adaptations to a transient gene expression workflow. The timeline of Example 2 is shown in the following Table 4:

| Day | Day | Day | Day | Day | Day | Day | Day | | Day |
|---|---|---|---|---|---|---|---|---|---|
| −2 | −1 | 0 | 1 | 2 | 3 | 4 | 5 | . . . | 14 |

| | | | | | |
|---|---|---|---|---|---|
| PIGGYBAC ® | Seed | Transfection | Bolus | Bolus | Harvest |
| PEI | cells | | feed | feed | |

Firstly, highly stringent selection for vector integration into transcriptionally-active sites within the host cell genome using MSX is not applied, although cultures are not supplemented with L-glutamine. Secondly, application of bolus feeds at days 2 and 5 post-transfection helps extend the culture and maximize yield towards the end of culture. Thirdly, the GS PIGGYBAC® transposase enzyme is co-transfected into the cells as circular plasmid DNA rather than as mRNA. As transiently transfected cultures are not intended for long-term cultivation or to generate clonal cell lines, inadvertent integration of the transposase gene into the genome is not considered to be a significant factor. Fourthly, PEI is used for transfection rather than electroporation.

### I. Preparation Prior to the Day of Transfection (Day −2)

1. Revive CHOK1SV GS-KO® host cell line from cryopreservation and perform routine culture until two days before transfection. In embodiments, CHOK1SV GS-KO® cells are in culture for no less than 4 days but no longer than 28 days before transfection. The culture has a viability of greater than 90% before transfection (as measured by trypan blue dye exclusion). Cultures are grown using CD CHO medium supplemented with 6 mM L-glutamine (CD CHO/6 mM glutamine) until before transfection.
2. Prepare the vector constructs for transfection.

Following preparation, the uncut GS vector DNA are suspended at 1000 g/mL in sterile Tris-EDTA (TE) buffer. The GS vectors contain the GS PIGGYBAC® ITR sequences to enable this method. In this example, the latest GSquad™ vector platform is used.
3. Two days before transfection, perform subculture of the host cell line:
3.1 Pre-warm the appropriate growth medium (CD CHO/6 mM glutamine) to 35.5-37.0° C.
3.2 Aseptically remove a sample of cell suspension from the CHOK1SV GS-KO® cells to be counted using a sterile serological pipette.
3.3 Use a haemocytometer or automated cell counting device to count the cells, using trypan blue dye or other method to assess cell viability. Use the cell count and viability data to determine the cell concentration and culture viability.
3.4 For each 20 mL of transiently transfected culture required, inoculate 30 mL of new culture at a target concentration of 0.2×10⁶ viable cells/mL. Larger transfection volumes can be achieved; a range of recommended culture volumes is given in the following Table 5:

| | Culture Volume to Inoculate |
|---|---|
| Final Culture Volume | the Day Before Transfection (Day −1) |
| Required (mL) | at 1.0 × 10⁶ viable cells/mL [a] (mL) |
| 20 | 30 |
| 40 | 60 |
| 100 | 150 |
| 120 | 300 |

3.5 If using vented-caps, re-fit the cap and place the new culture into a humid CO₂ orbital shaker incubator set at 35.5-37.0° C., 140±5 rpm, >85% relative humidity and 5% CO₂ in air.
3.6 If using sealed-caps, use a sterile, premixed supply of 5% CO₂ in air to gas out the headspace before re-fitting the cap and placing the new culture into an orbital shaker incubator set at 35.5-37.0° C. and 140±5 rpm.

### II. Transfection (Day 0)

On the day of transfection (two days after subculture, considered as Day 0 in this procedure):
1. Calculate the total amounts of medium, PEI, cells and DNA (both the GS PIGGYBAC®-compatible vector containing the product genes, and also the transposase-encoding plasmid SPB-DNA), that are needed for transfection.

A single transfection at a 20 mL scale requires a total of 20×10⁶ viable cells, 40 μg GS vector DNA, 4 μg SPB-DNA, and 100 μL PEI (Transporter 5). Transfections may be scaled up or down, but the relative ratios of reagents per mL of culture suitably should be maintained. Table 6 below shows ratios of reagents at transfection:

| | Amount per mL of | |
|---|---|---|
| Component | transfected culture | |
| CHOK1SV GS-KO ® host cells | 1 × 10⁶ viable cells | |
| Transporter 5 ® Transfection Reagent | 5 | μL |
| SPB-DNA(transposase plasmid; stock 1 mg/mL) | 0.2 | μg |
| Product vector (GS PIGGYBAC ®-compatible) | 2 | μg |

2. Aseptically prepare the appropriate volume of CD CHO with 10 mL/L HT supplement and pre-warm to 35.5-37.0° C.
3. Using a sterile serological pipette, aseptically remove a sample of cell suspension from the CHOKISV GS-KO® culture that had been inoculated at 0.2×10⁶ viable cells/mL two days before.
4. Use a haemocytometer or automated cell counting device to count the cells, using trypan blue dye or other method to assess cell viability. Use the cell count and viability data to determine the cell concentration, culture viability and total number of viable cells. Please note that the culture should be at least 90% viable. Poor viability will reduce the transfection efficiency.
5. Calculate the volume of culture needed to give the required number of cells for transfection (20×10⁶ viable cells per 20 mL transfection). Please note that cells can be lost during the centrifuging and washing steps. It is therefore advisable in the next step to centrifuge up to 2-fold more cells than are required and to centrifuge cells at 200×g.
5.1 Aseptically remove the calculated volume of culture (from the previous step) and centrifuge at 200×g for 5 minutes.
5.2 Remove the supernatant, dislodge the pellet by gently tapping the base of the tube, and disperse the cell pellet in a sufficient volume of pre-warmed CD CHO+10 mL/L HT supplement to give a concentration 1.0×10⁶ viable cells/mL.
5.3 Determine the viable cell concentration and adjust to a final concentration of 1.0×10⁶ viable cells/mL using CD CHO+10 mL/L HT supplement (sodium hypoxanthine (10 mM) and thymidine (1.6 mM), Thermo Fisher catalogue number 11067030).
6. For a single standard transfection, transfer 20 mL of the cell suspension from the previous step into a 125 mL Erlenmeyer flask.
7. To the flask add the following in the stated order, shaking vigorously after each addition:
7.1 40 μL (=40 μg) of sterile circular plasmid DNA encoding the product genes at a concentration of 1 mg/mL;
7.2 4 μg SPB-DNA circular plasmid DNA;
7.3 100 μL of PEI.
8. Place the cells back into the humid CO₂ orbital shaker incubator set at 35.5-37.0° C., 140±5 rpm, >85% relative humidity and 5% CO₂ in air. If using sealed-caps, use a sterile, premixed supply of 5% CO₂ in air to gas out the headspace before re-fitting the cap and placing the new culture into an orbital shaker incubator set at 35.5-37.0° C. and 140±5 rpm.
9. At days 2 and 5 post-transfection add 10% v/v (=2 mL for a 20 mL total transfection volume) of CHO CD EfficientFeed™ B Liquid Nutrient Supplement (Thermo Fisher catalogue number A 1024001).
10. Cultures may be periodically monitored for growth and viability using a haemocytometer or automated cell counting device. Please note that cultures should be harvested before viability drops below 90%.
11. After 14 days, harvest the cultures using a suitable method, remove samples from the culture vessels, clarify and store the culture supernatant at an appropriate temperature for further assays.

Please note that the expression vector and the transposase are used together to transfect the host cell line using PEI transfection reagent in the Example 2.

### Example 3: Titer of the Transposase Optimized Transient Expression

In this example, protein expression by the transposase optimized transient expression is performed as described in Example 2, and the result is compared with that of legacy platform (similar to that described in Example 1 but used electroporation for transfection) and that of a third party system.

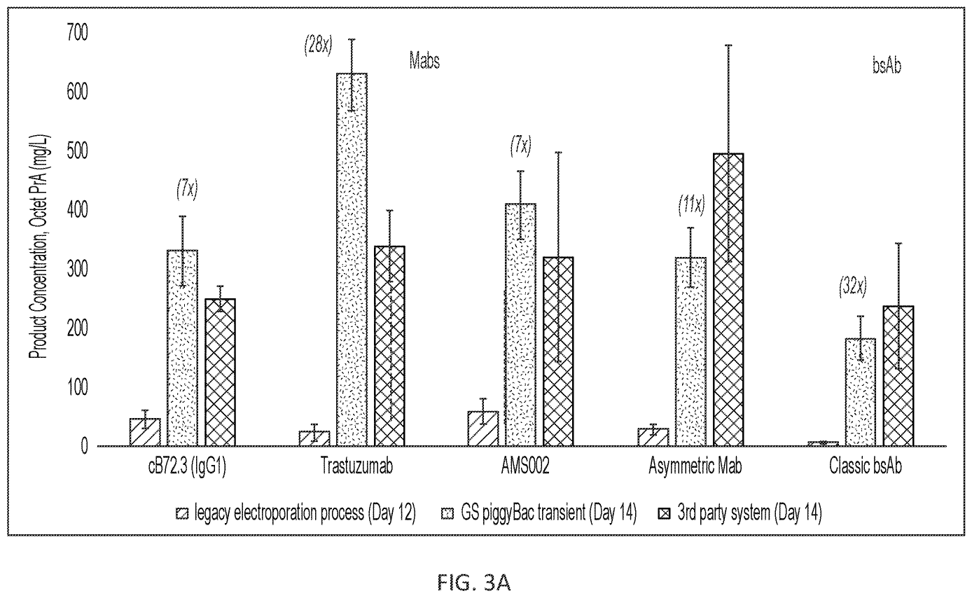

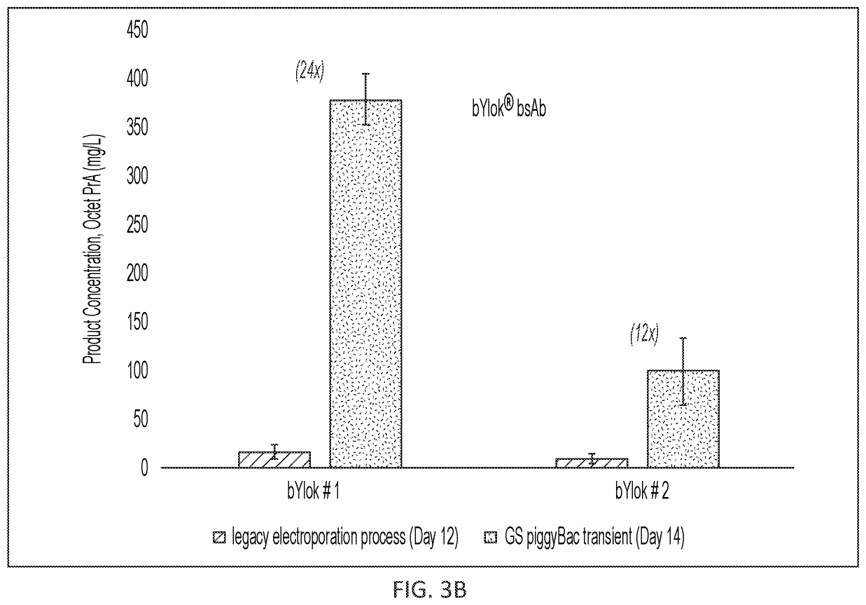

FIG. 3A shows comparison of the GS PIGGYBAC® transposase technology in transient expression with the legacy electroporation process and the third party system. The proteins of interest include cB72.3 (IgG1), Trstuzumab, AMS002, Asymmetric Mab, and Classic bsAb. As shown in FIG. 3A, using GS PIGGYBAC®, the transient titer is increased to up to 30 fold compared to that of the legacy process.

FIG. 3B shows comparison of the GS PIGGYBAC® transposase technology in transient expression with the legacy electroporation expression process to express bYlok® bsAb. The average harvest titer of the GS PIGGYBAC® transposase technology in transient expression is about 336 mg/L.

In conclusion, the optimized transient transfection process built around GS PIGGYBAC® achieves substantial titer increases in comparison to the legacy electroporation process, and is superior to third party systems under most of the conditions.

### Example 4: Product Quality of the Transposase Optimized Transient Expression

In this example, protein expression by the transposase optimized transient expression is performed as described in Example 2, and the product quality and attribute are analyzed. Particularly, GS PIGGYBAC® transposase technology is used in transient expression, as described in Example 2. Identical Xceed GS-KO® host cell line is used in both optimized transient expression and stable expression, and the protein of interest is cB72.3 (IgG1). The products from the two processes are compared.

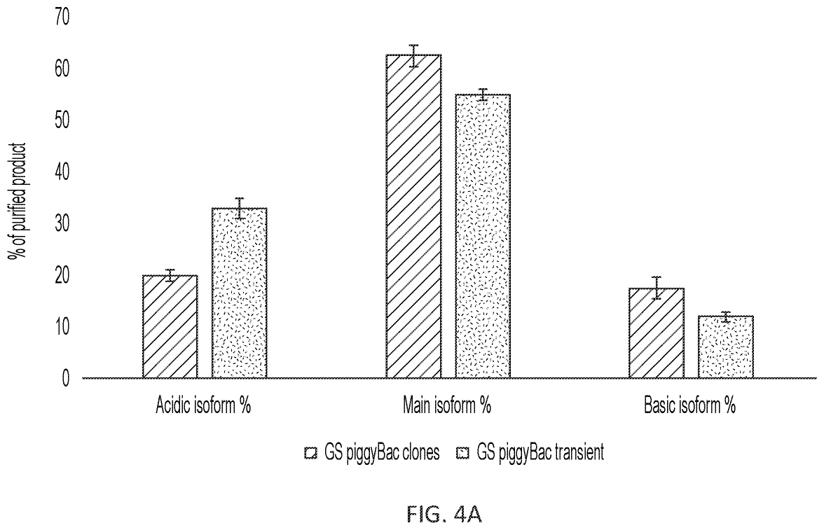

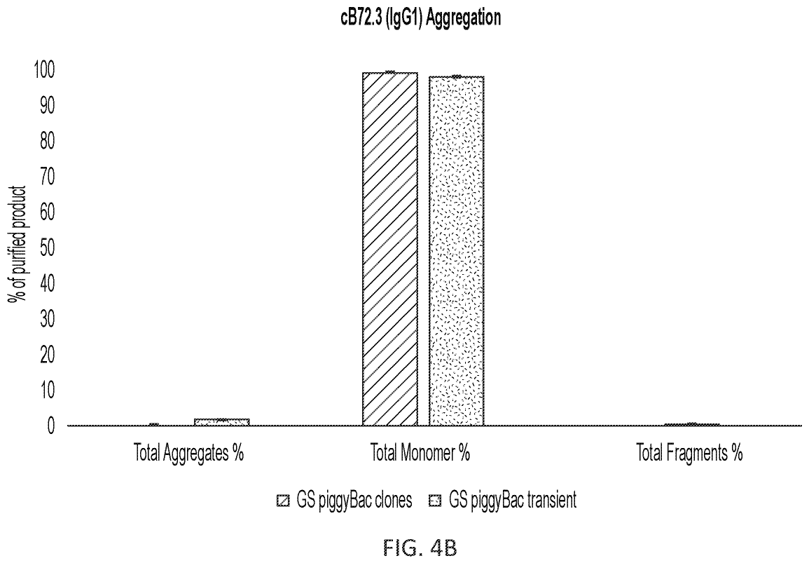

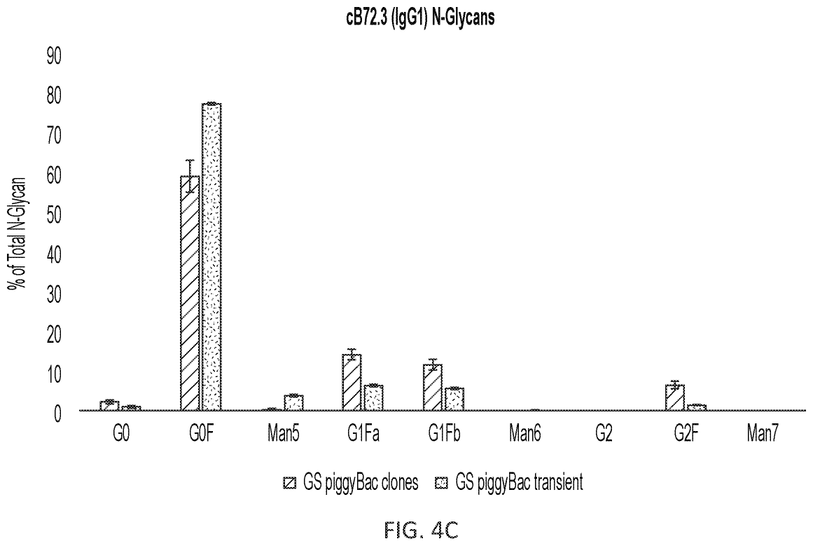

FIG. 4A shows isoforms of the cB72.3 products, FIG. 4B shows aggregation of the cB72.3 products, and FIG. 4C shows N-glycans of the cB72.3 products. The quality of the product from the optimized transient expression is comparable to that from the stable expression.

### Example 5: Determining Between +/−GS PIGGYBAC® Transient Transfection Process

This example provides a method of determining an appropriate transient process based on the balance of time, resource needed, and harvest titer needed. The +/−GS PIGGYBAC® transient transfection processes are performed as described in Example 2 and Example 1, respectively.

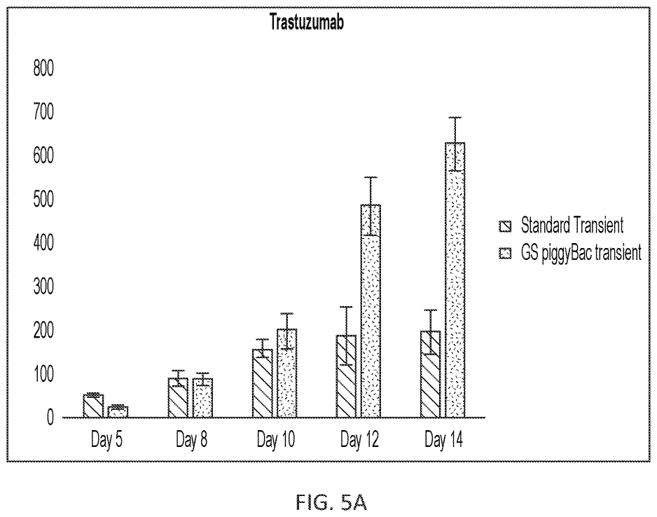

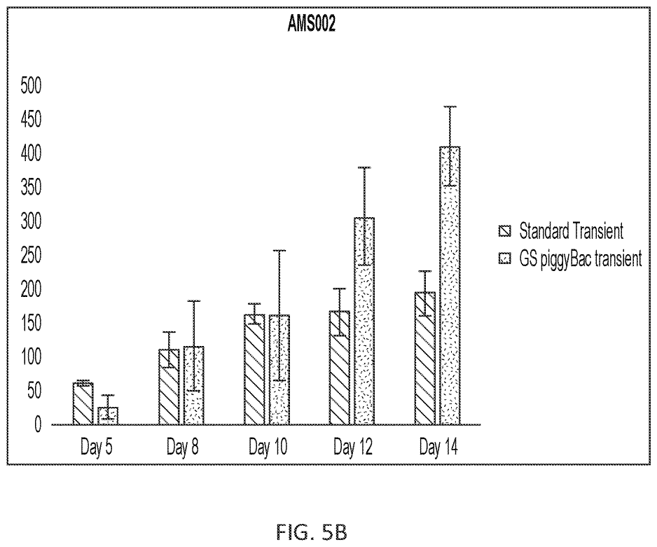

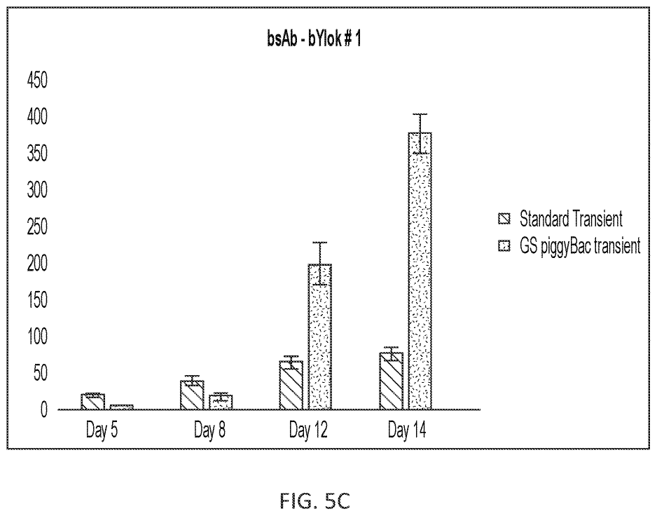

FIG. 5A shows expression of Trastuzumab using −GS PIGGYBAC® transient transfection process (standard transient) and +GS PIGGYBAC® transient transfection process (GS PIGGYBAC® transient). FIG. 5B shows the expression of AMS002. FIG. 5C shows expression of bsAb-bYlok #1.

The above result is summarized in the following Table 7:

| | Standard Transient | GS PIGGYBAC ® Transient |
|---|---|---|
| Harvest Titer | Lower | Higher |
| Duration | Shorter | Longer |
| Resource | Less | Greater |

In conclusion, the GS PIGGYBAC® Transient expression achieves highest titers with extended culture, while the standard process priorities speed/simplicity for rapid turnaround.

In summary, transient expression does not require a separate selection and expansion step after transfection, and the transfected cells are used directly for protein production. Although the speed is fast and the cost is low, the product provided by transient expression has a low titer. Stable pool expression requires a selection and expansion step after transfection and before production. The selection condition is stringent, and passage of cells is needed. Although the product titer is high, stable pool expression has a slow speed and has a high cost. The transposase optimized transient expression of the present disclosure balances speed, cost and product yield, where the selection/expansion and the production are performed simultaneously, low stringent selection is applied, no cell passage is required. The entirety of the transfected cell culture is directly and immediately moved into a product production phase wherein no cells are removed by any intermediate sub-culture step. As a result, the transposase optimized transient expression achieves high yield of product within time extended from transient expression, while shorter than that of the stable pool expression.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

What is claimed is:

1. A method of producing a protein of interest, comprising:
providing a host cell, a vector comprising a nucleic acid sequence encoding the protein of interest, and a transposase;
transfecting the host cell using the vector and the transposase to obtain a transfected cell;
culturing the transfected cell to form a culture; and
harvesting the culture to obtain the protein of interest from the harvested culture,
wherein cells of the culture are not passaged prior to harvesting the culture.

2. The method of claim 1, wherein the host cell comprises a mammalian cell line.

3. The method of claim 2, wherein the mammalian cell line comprises a Chinese hamster ovary (CHO) cell line, a baby hamster kidney (BHK) cell line, a murine myeloma cell line, a human embryonic kidney cell line (HEK) cell line, or a HeLa cell line.

4. The method of claim 1, wherein the host cell comprises a CHOK1 SV GS-knock out cell line.

5. The method of any one of claims 1 to 4, wherein the host cell is pre-cultured for about 4 days to about 28 days to form a pre-culture before the transfecting.

6. The method of claim 5, wherein the host cell is pre-cultured in a chemically defined Chinese hamster Ovary (CD CHO) medium supplemented with 6 mM glutamine before transfection.

7. The method of claim 5 or claim 6, wherein the pre-culture has at least 90% cell viability.

8. The method of any one of claims 1 to 7, wherein the vector comprises an adenoviral vector, a pSV vector, a pCMV vector, a vaccinia vector, a retroviral vector, or a baculovirus vector.

9. The method of any one of claims 1 to 8, wherein the vector comprises a glutamine synthetase gene.

10. The method of claim 9, wherein the vector comprises a hyperactive super PIGGYBAC® inverted terminal repeat (ITR) sequence.

11. The method of any one of claims 1 to 10, wherein the vector comprises one to about 5 nucleic acid sequences encoding the protein of interest, or the vector comprises one to about 5 different vectors comprising the nucleic acid sequence.

12. The method of any one of claims 1 to 11, wherein the transposase comprises an RNase H-like transposase, a HUH single-stranded DNA transposase, a serine transposase, a tyrosine transposase, a PIGGYBAC® transposase, or a combination thereof.

13. The method of claim 12, wherein the transposase comprises a PIGGYBAC® transposase.

14. The method of any one of claims 1 to 13, wherein the transposase is contained in a transposase vector or delivered as mRNA.

15. The method of any one of claims 1 to 14, wherein transfecting the host cell is performed using polyethylenimine (PEI), lipofection, electroporation, magnetofection, microinjection, gene gun insertion, impalefection, hydrostatic pressure, or sonication.

16. The method of claim 15, wherein transfecting the host cell is performed using PEI.

17. The method of any one of claims 1 to 16, wherein the transfected cells are cultured in a medium, and the medium comprises Dulbecco's Modified Eagle Medium (DMEM), Roswell Park Memorial Institute (RPMI) 1640, Minimum Essential Medium (MEM), a chemically defined Chinese hamster Ovary (CD CHO) medium without supplementing glutamine, or a combination thereof.

18. The method of claim 17, wherein the medium comprises CD CHO medium without supplementing glutamine.

19. The method of claim 18, wherein the culture is devoid of methionine sulfoxamine (MSX).

20. The method of any one of claims 1 to 19, wherein the culture is bolus fed at day 2 and day 5 after transfecting the host cell.

21. The method of any one of claims 1 to 20, wherein transfecting occurs on day 1 and harvesting begins on day 2 of the method.

22. The method of claim 21, wherein harvesting continues from day 2 to at least day 14 of the method.

23. A method of producing a protein of interest, comprising:
providing a host cell, a vector comprising a nucleic acid sequence encoding the protein of interest, and a transposase;
transfecting the host cell using the vector and the transposase to obtain a transfected cell;
culturing the transfected cell to form a culture; and
harvesting the culture to obtain the protein of interest from the harvested culture,
wherein cells of the culture are of the same cell lineage as the transfected cell.

24. The method of claim 23, wherein the host cell comprises a mammalian cell line.

25. The method of claim 24, wherein the mammalian cell line comprises a Chinese hamster ovary (CHO) cell line, a baby hamster kidney (BHK) cell line, a murine myeloma cell line, a human embryonic kidney cell line (HEK) cell line, or a HeLa cell line.

26. The method of claim 23, wherein the host cell comprises a CHOK1SV GS-knock out cell line.

27. The method of any one of claims 23 to 26, wherein the host cell is pre-cultured for about 4 days to about 28 days to form a pre-culture before the transfecting.

28. The method of claim 27, wherein the host cell is pre-cultured in a chemically defined Chinese hamster Ovary (CD CHO) medium supplemented with 6 mM glutamine before transfection.

29. The method of claim 27 or claim 28, wherein the pre-culture has at least 90% cell viability.

30. The method of any one of claims 23 to 29, wherein the vector comprises an adenoviral vector, a pSV vector, a pCMV vector, a vaccinia vector, a retroviral vector, or a baculovirus vector.

31. The method of any one of claims 23 to 30, wherein the vector comprises a glutamine synthetase gene.

32. The method of claim 31, wherein the vector comprises a hyperactive super PIGGYBAC® inverted terminal repeat (ITR) sequence.

33. The method of any one of claims 23 to 32, wherein the vector comprises one to about 5 nucleic acid sequences encoding the protein of interest, or the vector comprises one to about 5 different vectors comprising the nucleic acid sequence.

34. The method of any one of claims 23 to 33, wherein the transposase comprises an RNase H-like transposase, a HUH single-stranded DNA transposase, a serine transposase, a tyrosine transposase, a PIGGYBAC® transposase, or a combination thereof.

35. The method of claim 34, wherein the transposase comprises a PIGGYBAC® transposase.

36. The method of any one of claims 23 to 35, wherein the transposase is contained in a transposase vector.

37. The method of any one of claims 23 to 36, wherein the transfecting the host cell is performed using polyethylenimine (PEI), lipofection, electroporation, magnetofection, microinjection, gene gun insertion, impalefection, hydrostatic pressure, or sonication.

38. The method of claim 37, wherein the transfecting the host cell is performed using PEI.

39. The method of any one of claims 23 to 38, wherein the transfected cells are cultured in a medium, and the medium comprises Dulbecco's Modified Eagle Medium (DMEM), Roswell Park Memorial Institute (RPMI) 1640, Minimum essential medium (MEM), a chemically defined Chinese hamster Ovary (CD CHO) medium without supplementing glutamine, or a combination thereof.

40. The method of claim 39, wherein the medium comprises CD CHO medium without supplementing glutamine.

41. The method of claim 40, wherein the culture is devoid of methionine sulfoxamine (MSX).

42. The method of any one of claims 23 to 41, wherein the culture is bolus fed at day 2 and day 5 after transfecting the host cell.

43. The method of any one of claims 23 to 42, wherein the transfecting occurs on day 1 and the harvesting begins on day 2 of the method.

44. The method of claim 43, wherein the harvesting continues from day 2 to at least day 14 of the method.

45. A method of producing a viral gene of interest, comprising:
providing a host cell, a vector comprising a nucleic acid sequence encoding the viral gene of interest, and a transposase;
transfecting the host cell using the vector and the transposase to obtain a transfected cell;
culturing the transfected cell to form a culture; and
harvesting the culture to obtain the viral gene of interest from the harvested culture,
wherein cells of the culture are not passaged prior to harvesting the culture.

46. The method of claim 45, wherein the viral gene of interest produces an AAV virus particle or a lentivirus particle.
